(19) United States
(12) Patent Application Publication
(10) Pub. No.: US 2019/0380631 A1
(43) Pub. Date: Dec. 19, 2019

(54) Methods and Systems for Low Power/Low Cost Hematocrit Measurement for Blood Glucose Meter

(71) Applicant: Trividia Health, Inc., Fort Lauderdale, FL (US)

(72) Inventors: Steven V. Leone, Lake Worth, FL (US); Brent E. Modzelewski, Boca Raton, FL (US)

(21) Appl. No.: 16/444,294

(22) Filed: Jun. 18, 2019

Related U.S. Application Data
(60) Provisional application No. 62/686,599, filed on Jun. 18, 2018.

(51) Int. Cl. A61B 5/145; G01N 33/66; G01N 33/49; G01N 33/487

(52) U.S. Cl. CPC A61B 5/14535; G01N 33/66; A61B 5/1486; G01N 33/48785; A61B 5/14532; G01N 33/4905

(57) ABSTRACT

Systems and methods for hematocrit measurement are provided. The present disclosure provides systems and methods for a low power and low-cost utilization of a microprocessor to capture hematocrit measurements for a blood glucose meter. The systems and methods include an electronic meter for performing a diagnostic test on a sample applied to a test strip inserted. The electronic meter can include a low cost low power microcontroller for capturing the hematocrit measurements. The microcontroller includes trans-impedance amplifiers (TIAs) configured to read a current over the test strip to generate a response waveform, an analog-to-digital (ADC) converter to convert the response waveform into a digital value, a peak detector circuit configured to capture peak current magnitude of the response waveform, and a peak detector circuit configured to capture a peak current time

### RELATED APPLICATIONS

This application claims priority to and the benefit of U.S. Provisional Patent Application No. 62/686,599, filed Jun. 18, 2018, the entire contents of which are incorporated herein by reference.

### FIELD

The present disclosure relates to systems and methods for low power/low cost hematocrit measurement in connection with blood glucose meters.

### BACKGROUND

Many industries have a commercial need to monitor the concentration of particular constituents in a fluid. In the health care field, for example, individuals with diabetes have a need to monitor a particular constituent within their bodily fluids. A number of systems are available that allow people to test a body fluid, such as, blood, urine, or saliva, to conveniently monitor the level of a particular fluid constituent, such as, cholesterol, proteins, and glucose. Such systems typically include a test strip where the user applies a fluid sample and a meter that “reads” the test strip to determine the level of the tested constituent in the fluid sample. A Blood Glucose Monitor (BGM) is an example of such a device.

Conventionally, a BGM is a portable handheld device used to measure blood glucose levels for users with Type I or Type II diabetes. Typically, the user purchases small strips (approximately 20-30 mm×5-9 mm) that interface with the BGM. The user draws a tiny amount of blood (a few microliters) from a finger or other area using a lancer, applies a blood droplet sample onto the exposed end of the strip, and then inserts the connector end of the strip into the BGM connector port. A chemical reaction occurs between the blood sample and the chemistry on the strip, which is measured by the BGM to determine the blood glucose level in units of mg/dL or mmol/L, or Kg/L depending on regional preferences.

Two resources that are constrained in Blood Glucose Meter (BGM) designs are energy and processing power. To keep the cost and size down, portable BGMs are typically powered by a small single CR2032 type coin cell Lithium battery or similar. The peak source current of this type of battery is very low and the total current capacity is also very low, from tens to a few hundred milli-Amp-hours (mAh). Yet, this small battery is expected to last the life of the meter, or at least require extremely infrequent battery changes. There is thus a need for low power methods and systems for measuring haematocrit to extend the life of the BGM batteries.

### SUMMARY

The present disclosure provides systems and methods for hematocrit measurement. In particular, the present disclosure provides systems and methods for obtaining a low power and low-cost hematocrit measurement for a blood glucose meter.

In accordance with example embodiments of the present disclosure, a system for diagnostic testing is provided. The system includes a test strip and an electronic meter for performing a diagnostic test on a sample applied to the test strip inserted therein. The electronic meter includes a housing having a test port for receiving the test strip, trans-impedance amplifiers (TIAs) configured to read a current over the test strip to generate a response waveform, an analog-to-digital (ADC) converter to convert the response waveform into a digital value, a peak detector circuit configured to capture peak current magnitude of the response waveform, and a peak detector circuit configured to capture a peak current time.

In accordance with aspects of the present disclosure, the reading the current over the test strip includes measuring a response current to an excitation voltage. The TIAs can generate the response waveform from the current. The peak detector circuit can capture the peak current time based on the captured peak current magnitude from the peak detector circuit and the response waveform from the TIAs. An excitation signal can be applied to the blood sample such that the response to the excitation signal is analyzed to determine a glucose concentration in the blood sample. The TIAs can convert current to a differential voltage waveform for analog-to-digital conversion by the ADC. A microprocessor can be further programmed to detect when the sample is applied to the test strip. A microprocessor can be further programmed to display a glucose concentration.

In accordance with example embodiments of the present disclosure, a diagnostic testing device is provided. The diagnostic testing device includes a housing having a test port for receiving a test strip, trans-impedance amplifiers (TIAs) configured to read a current over the test strip to generate a response waveform, and a peak detector circuit configured to capture peak current magnitude of the response waveform. The diagnostic testing device further includes a peak detector circuit configured to capture a peak current time and an analog-to-digital (ADC) converter to convert the response waveform and the peak current magnitude into a digital value.

In accordance with aspects of the present disclosure, the reading the current over the test strip includes measuring a response current to an excitation voltage. The TIAs can generate the response waveform from the current. The peak detector circuit can capture the peak current time based on the captured peak current magnitude from the peak detector circuit and the response waveform from the TIAs. An excitation signal can be applied to the blood sample such that the response to the excitation signal is analyzed to determine a glucose concentration in the blood sample. The TIAs can convert current to a differential voltage waveform for analog-to-digital conversion by the ADC. A microprocessor can be further programmed to detect when the sample is applied to the test strip. A microprocessor can be further programmed to display a glucose concentration.

A method for a low cost and low power hematocrit testing is provided. The method includes applying a voltage to a sample on a test strip, measuring, by trans-impedance amplifiers (TIAs), a current through the sample, processing, by the TIAs, the current and outputting a voltage response waveform to an analog digital converter (ADC), processing, by the peak detector, the voltage response waveform and outputting a peak current to the ADC, and calculating and displaying hematocrit compensated glucose results based on the values received by the ADC.

In accordance with aspects of the present disclosure, method further includes outputting, by the TIAs, the voltage response waveform to a peak comparator and outputting, by the peak detector, the peak current to the peak comparator. The method can further include processing, by the peak comparator, the voltage response waveform and the peak current and outputting a peak time value. The calculating the compensated glucose results can further include calculating an HTC value based on the peak current and peak time value; and adjusting a glucose measurement to the hematocrit compensated glucose results.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1A is a general cross-sectional view of a test strip according to some embodiments of the present disclosure;

FIG. 1B is a top view of a conductive pattern on a substrate of a test strip according to some embodiments of the present disclosure;

FIGS. 2A and 2B illustrate a meter according to some embodiments of the present disclosure;

FIG. 3 is an exemplary a block diagram for a Blood Glucose Monitor utilizing a low power, low cost hematocrit (HCT) measurement technique;

FIG. 4 is an exemplary block diagram for a Transimpedance Amplifier (TIA) for HCT electrode;

FIG. 5 is an equivalent circuit model of blood sample;

FIG. 6 is a graph showing current through a blood sample that is matched to the equitant circuit of FIG. 4 as part of a step response;

FIG. 7 is an exemplary block diagram for an embodiment of a peak detector used to hold the peak value for peak current and as input to peak time comparator;

FIG. 8 is an exemplary block diagram for an embodiment of a comparator used to capture a peak time;

FIG. 9 is a plot of the circuit waveforms for peak current and peak time;

FIG. 10 is an exemplary flow chart for an operation of the present disclosure;

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure provides systems and methods for implementing a low power and low-cost microcontroller for obtaining hematocrit measurements. Such hematocrit measurements can be obtained using time domain linear system techniques to detect key parameters of a step response signal on a set of hematocrit electrodes. These parameters can then be utilized to compute blood glucose and hematocrit compensation from a blood sample on a chemistry strip.

In order to determine a measurement of an analyte, such as blood glucose, in a sample, such as blood, using a device, such as a blood glucose meter, certain interferents can be accounted for to increase the accuracy of the measurement. For example, one such interferent is the hematocrit (HCT) concentration in the blood. In some embodiments, a method of measuring the HCT for a blood glucose meter using a peak current detector and a peak current time comparator can be utilized. The percent of HCT concentration can be mapped to the HCT peak current and peak current time to measure the concentration of the analyte, such as glucose, in the blood.

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments

Specific details are given in the following description to provide a thorough understanding of the embodiments. However, it will be understood by one of ordinary skill in the art that the embodiments may be practiced without these specific details. For example, systems, processes, and other elements in the presently disclosed embodiments may be shown as components in block diagram form in order not to obscure the embodiments in unnecessary detail. In other instances, well-known processes, structures, and techniques may be shown without unnecessary detail to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed, but could have additional steps not discussed or included in a figure. Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function.

Subject matter will now be described more fully with reference to the accompanying drawings, which form a part hereof, and which show, by way of illustration, specific example aspects and embodiments of the present disclosure. Subject matter may, however, be embodied in a variety of different forms and, therefore, covered or claimed subject matter is intended to be construed as not being limited to any example embodiments set forth herein; example embodiments are provided merely to be illustrative. The following detailed description is, therefore, not intended to be taken in a limiting sense.

A meter for measuring blood glucose or another analyst can include a portable, handheld device used to measure blood glucose levels for users with Type I or Type II diabetes. Typically, the user purchases test strips that interface with the meter. The user draws a tiny amount of blood (a few microliters or less) from a finger or other area using a lancer and a blood droplet is applied onto the exposed end of the strip which has an open port for the blood. The strip is inserted into the meter connector port and a chemical reaction occurs between the blood sample and the chemistry on the strip, which is measured by the meter to determine the blood glucose level in units of mg/dL or mmol/L, depending on regional preferences.

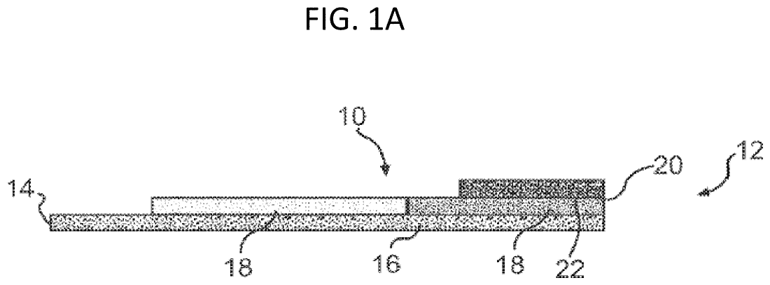

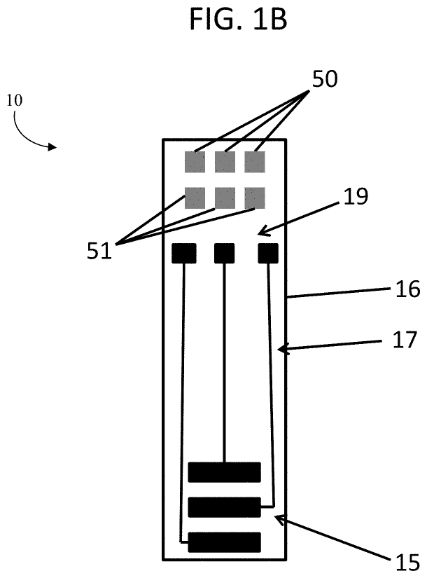

FIG. 1A illustrates a general cross-sectional view of an example embodiment of a test strip 10. In particular, FIG. 1A depicts a test strip 10 that includes a proximal end 12, a distal end 14, and is formed with a base layer 16 extending along the entire length of test strip 10. The base layer 16 is preferably composed of an electrically insulating material and has a thickness sufficient to provide structural support to test strip 10. For purposes of this disclosure, “distal” refers to the portion of a test strip further from the fluid source (e.g., closer to the meter) during normal use, and “proximal” refers to the portion closer to the fluid source (e.g., a fingertip with a drop of blood for a glucose test strip) during normal use. The base layer 16 may be composed of an electrically insulating material and has a thickness sufficient to provide structural support to test strip 10.

As seen in FIG. 1A, the proximal end 12 of test strip 10 includes a sample receiving location, such as a sample chamber 20 configured to receive a patient's fluid sample, as described above. The sample chamber 20 may be formed in part through a slot in a dielectric insulating layer 18 formed between a cover 22 and the underlying measuring electrodes formed on the base layer 16. Accordingly, the sample chamber 20 may include a first opening, e.g., a sample receiving location, in the proximal end of the test strip and a second opening for venting the sample chamber 20. The sample chamber 20 may be dimensioned to be able to draw the blood sample in through the first opening, and to hold the blood sample in the sample chamber 20, by capillary action. The test strip 10 can include a tapered section that is narrowest at the proximal end 12, or can include other indicia to make it easier for the user to locate the first opening and apply the blood sample.

In accordance with an example embodiment of the present disclosure, the strip 10 can include a conductive pattern. In reference to FIG. 1B, disposed on base layer 16 of the strip 10 is a conductive pattern. In some embodiments, the conductive pattern may be formed by laser ablating the electrically insulating material of the base layer 16 to expose the electrically conductive material underneath. Other methods may also be used, such as inserted conductors with physical attachment to control circuit. Other methods may also be used to dispose the conductive pattern on the base layer. The conductive pattern may include a plurality of electrodes 15 disposed on base layer 16 near proximal end 12, a plurality of electrical strip contacts 19 disposed on base layer 16 near distal end 14, and a plurality of conductive traces 17 electrically connecting the electrodes 15 to the plurality of electrical strip contacts 19.

In accordance with an example embodiment of the present disclosure, a reagent layer may be disposed on the base layer 16 of the strip 10 in contact with at least a working electrode of the conductive pattern. The reagent layer may include an enzyme, such as glucose oxidase, and a mediator, such as potassium ferricyanide or ruthenium hexamine. Reagent layer 90 may also include other components, such as buffering materials (e.g., potassium phosphate), polymeric binders (e.g., hydroxypropyl-methyl-cellulose, sodium alginate, microcrystalline cellulose, polyethylene oxide, hydroxyethylcellulose, and/or polyvinyl alcohol), and surfactants (e.g., Triton X-100 or Surfynol 485). With these chemical constituents, the reagent layer reacts with glucose in the blood sample in the following way. The glucose oxidase initiates a reaction that oxidizes the glucose to gluconic acid and reduces the ferricyanide to ferrocyanide. When an appropriate voltage is applied to working electrode, relative to counter electrode, the ferrocyanide is oxidized to ferricyanide, thereby generating a current that is related to the glucose concentration in the blood sample. As would be appreciated by one skilled in the art, any combination of strips 10 known in the art can be utilized without departing from the scope of the present disclosure.

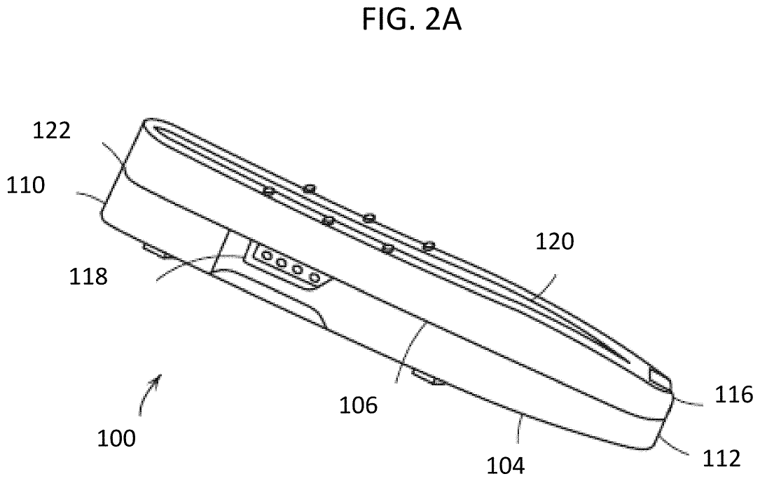

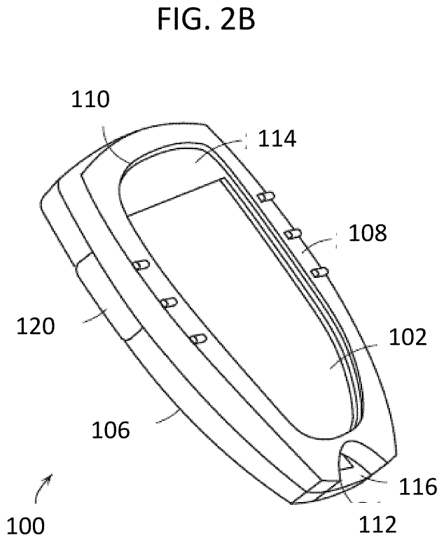

FIG. 2A and FIG. 2B illustrate an exemplary illustration of a meter 100 used to measure the glucose level in a blood sample. The meter 100 includes a housing having a test port for receiving the test strip, and a processor or microprocessor programmed to perform methods and algorithms to determine glucose concentration in a test sample or control solution as disclosed in the present disclosure. In some embodiments, the meter 100 has a size and shape to allow it to be conveniently held in a user's hand while the user is performing the glucose measurement. The meter 100 may include a front side 102, a back side 104, a left side 106, a right side 108, a top side 110, and a bottom side 112. The front side 102 may include a display 114, such as a liquid crystal display (LCD). A bottom side 112 may include a strip connector 116 into which test strip can be inserted to conduct a measurement. The meter 100 may also include a storage device for storing test algorithms or test data. The left side 106 of the meter 100 may include a data connector 418 into which a removable data storage device 120 may be inserted, as necessary. The top side 110 may include one or more user controls 122, such as buttons, with which the user may control meter 100, and the right side 108 may include a serial connector (not shown).

In some embodiments, the blood glucose meter comprises a decoder for decoding a predetermined electrical property, e.g. resistance, from the test strips as information. The decoder operates with, or is a part of, the microprocessor.

The meter can be programmed to wait for a predetermined period of time after initially detecting the blood sample, to allow the blood sample to react with the reagent layer or can immediately begin taking readings in sequence. During a fluid measurement period, the meter applies an assay voltage between the working and counter electrodes and takes one or more measurements of the resulting current flowing between the working and counter electrodes. The assay voltage is near the redox potential of the chemistry in the reagent layer, and the resulting current is related to the concentration of the particular constituent measured, such as, for example, the glucose level in a blood sample.

In one example, the reagent layer may react with glucose in the blood sample to determine the particular glucose concentration. In one example, glucose oxidase is used in the reagent layer. The recitation of glucose oxidase is intended as an example only and other materials can be used without departing from the scope of the disclosure. Other possible mediators include, but are not limited to, ruthenium and osmium. During a sample test, the glucose oxidase initiates a reaction that oxidizes the glucose to gluconic acid and reduces the ferricyanide to ferrocyanide. When an appropriate voltage is applied to a working electrode, relative to a counter electrode, the ferrocyanide is oxidized to ferricyanide, thereby generating a current that is related to the glucose concentration in the blood sample. The meter then calculates the glucose level based on the measured current and on calibration data that the meter has been signaled to access by the code data read from the second plurality of electrical contacts associated with the test strip. The meter then displays the calculated glucose level to the user.

A correction based on a measured HCT value can be applied to glucose level determined by the meter. In some embodiments, the HCT measurement sequence begins after a drop of blood or control is detected when the drop completes the circuit between the HCT measurement anode and cathode. In some embodiment, the HCT is analysed based on an electrical measurement between two electrodes on the test strip separate from the electrodes used to measure glucose, or the electrodes can be shared for both measurements. After the drop is detected an excitation voltage signal is applied to the HCT electrodes. The salt content of blood creates an electronic signature, in which the magnitude and phase response can be mapped to the HCT of the blood. The impedance of the electrical signature is affected by temperature, so the true HCT reading is corrected for temperature for the temperature difference from 24° C. (dT).

In some embodiments, the glucose measurement sequence is initiated only when the meter detects a full sample chamber. The glucose in the test sample is oxidized by the enzyme glucose dehydrogenase-FAD, producing gluconolactone and the reduced form of an electron mediator. The reduced mediator is then oxidized at the surface of the glucose measurement anode to produce an electrical signal (current in nanoamp units) that is detected by the meter. The electrical signal (current, in nanoamps) produced by oxidation of the reduced mediator at the surface of the glucose measurement anode is proportional to the amount of glucose in the test sample. The HCT value (which can be temperature corrected) is then used to determine the temperature corrected glucose value.

The meter can measure blood glucose by analysing the electrical response to an excitation signal. However, this response is dependent on the HCT concentration in the blood. The accuracy of the glucose measurement is therefore dependent on the accuracy of the HCT concentration to compensate the measurement for this interferent. For a given blood glucose sample, the peak response current to a voltage excitation used to measure blood glucose on the blood sample can be inversely proportional to the HCT concentration in the blood. Knowing the HCT peak current provides the data to map the HCT concentration to the peak current through empirical methods. This known HCT concentration (% HCT), can then be used to provide an accurate blood glucose measurement.

Various systems and methods can be used for measuring the HCT concentration from step response to impedance measurement. In some embodiments, a low cost and low power microcontroller can be utilized to compute blood glucose and hematocrit compensation from a blood sample on a chemistry strip. Because handheld meters typically rely on a small battery such that use of the low power microcontroller can be required for the blood glucose algorithm computations. Low power microcontrollers are limited in their maximum operating frequency (typically 1-8 MHz), and the small battery cannot support more than a few milliamperes during the blood glucose computation without drooping significantly and causing a system reset. However, because of this low operating frequency, the sampling rate on the analog to digital converter (ADC) is also limited to a maximum rate much less than would be required to fully re-construct all the blood measurements (hematocrit (HCT) in particular).

Specifically, the initial transient generated on the HCT signal from a step response includes the capture of peak current magnitude and time of peak current, which occurs much faster than the maximum sampling rate. These peak parameters, in addition to the current decay rate are needed to accurately reconstruct the parameters of the equivalent circuit which will yield the blood glucose/hematocrit values. The relatively slow decay rate can be accurately sampled with the slower ADC beyond the initial peak transient rising edge.

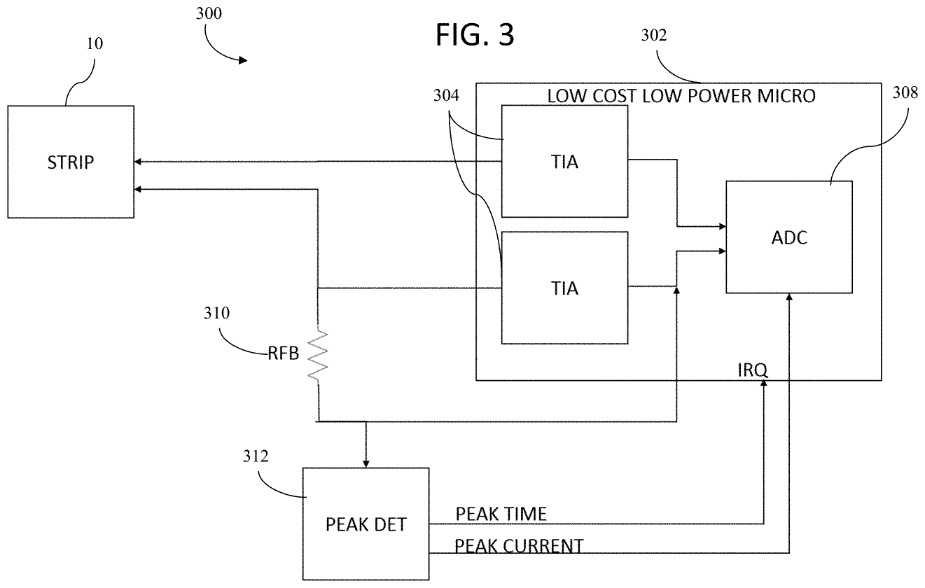

The present disclosure provides systems and methods for implementing a low power and low-cost microcontroller for obtaining hematocrit measurements. In some embodiments, the hematocrit measurements can be obtained using time domain linear system techniques to detect key parameters of a step response signal on a set of hematocrit electrodes to compute blood glucose and hematocrit compensation from a blood sample (e.g., on a chemistry strip). FIG. 3 illustrates an example embodiment of a system 300 for implementing a low cost and low power microcontroller 302 for use with a BGM (such as the BGM 100). In particular, FIG. 3 depicts a block diagram of a system 300 including a circuit illustrating an analog front end (AFE) for a low cost, low power microcontroller 302 for use in measuring hematocrit for a BGM. In some embodiments, the AFE can include two integrated trans-impedance amplifiers (TIA) 304 and an analog-to-digital converter (ADC) 308. The microcontroller 302 can include the TIAs 304 for test strip current measurements and the ADC 308 to sample the analog signals from the TIAs 304 and convert them into digital values. In operation, the TIA 304 can be configured to convert current to differential voltage signal for an ADC conversion by the ADC 308. In some embodiments, the system 300 can include a peak detector circuit 312 configured to detect a peak current and capture a time of the peak by introducing a step voltage to a sample (e.g., blood sample) on the strip 10 and the system 300.

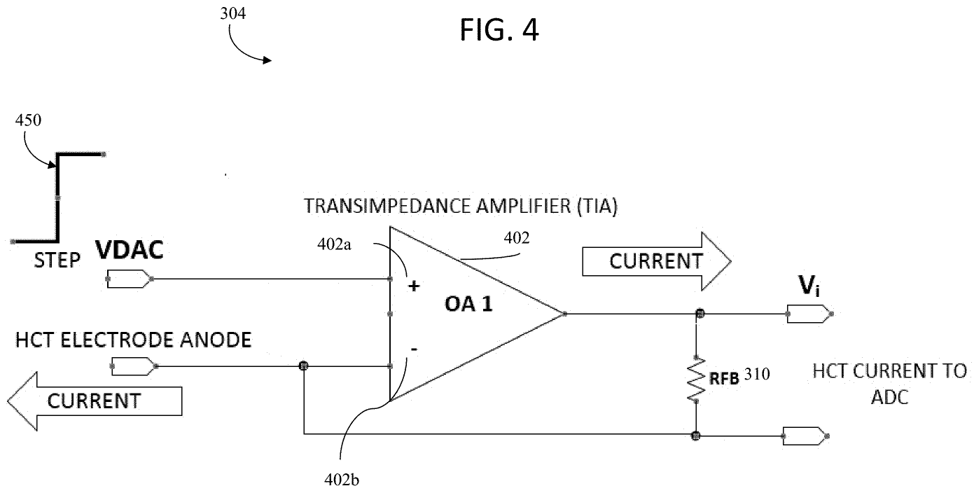

FIG. 4 depicts a detailed view of an exemplary depiction of a TIA 304 for the HCT electrode of the microcontroller 302 of FIG. 3. In particular, FIG. 4 depicts a TIA 304 connected to a resistance feedback (RFB) circuit 310 and the ADC 308. The TIA 304 includes an operational amplifier 402 that can convert current to a differential analog voltage for input to the analog to digital converter 308. In some embodiments, the digital to analog converter 308 can use the input to produce a step rising edge excitation (such as shown by rising edge 450) at the non-inverting input terminal 402a of the operational amplifier 402. Voltage from the RFB 310 and the HCT electrode anode on the inverting input terminal 402b operational amplifier 402 represent the current as a voltage to the ADC 308.

In operation, in some embodiments, a voltage step response 450 can be generated by an action (e.g., a drop of blood via a strip 10) that completes the circuit between the HCT measurement anode and cathode of a meter (e.g., meter 100) and can then be input on the non-inverting input terminal 402a of the operational amplifier 402. For example, when a drop of blood is detected, an excitation voltage signal can be applied to the HCT electrodes connected to the TIA 304 and a current can be generated by the TIA 304 for input into the ADC 308. The response to the step response 450 input can also be the current that is output from the operational amplifier 402 across the RFB 310. Current measurement of the non-inverting operational amplifier 402 can be achieved by passing the sense current across the feedback resistor 310 on the inverting input terminal 402b, via the RFB 310, to produce a differential voltage drop across RFB 310 that is proportional to the current being sensed. Additionally, current (e.g., the HCT current) is output from the operational amplifier across the RFB 310 to the ADC 308. In some embodiments, the value of RFB 310 is chosen such that the voltage developed across the RFB 310 to the ADC 308 utilizes the full range of the ADC 308 but not more as in the equation RFB max<(VADCmax/I peak). In some embodiments, the microcontroller 302 includes two TIA 304 operating in parallel, with one TIA 304 dedicated to glucose current measurement and the other TIA 304 to the HCT measurement.

In some embodiments, the system 300 can be implanted as part of a BGM device (e.g., meter 100) configured to obtaining a reading (e.g., an HCT reading) from a blood sample on a strip 10. As noted above, after the voltage drop is detected, an excitation voltage signal can be applied to the HCT electrodes. The salt content of blood creates an electronic signature as a reading, in which the magnitude and phase response can be mapped to the HCT of the blood.

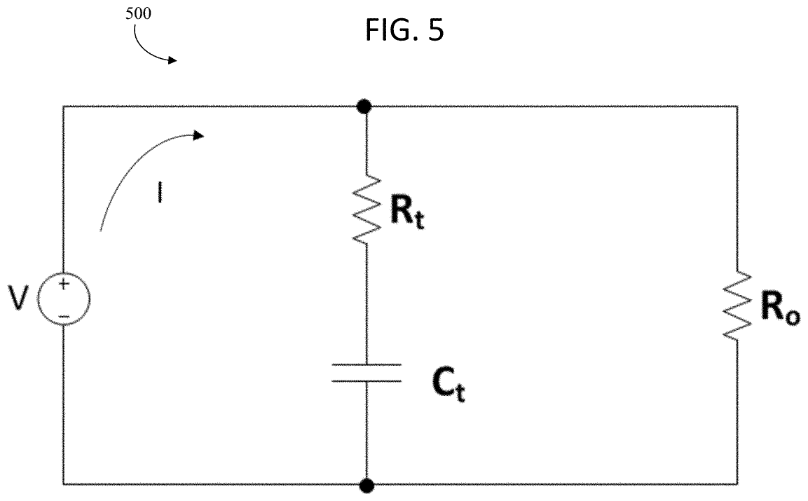

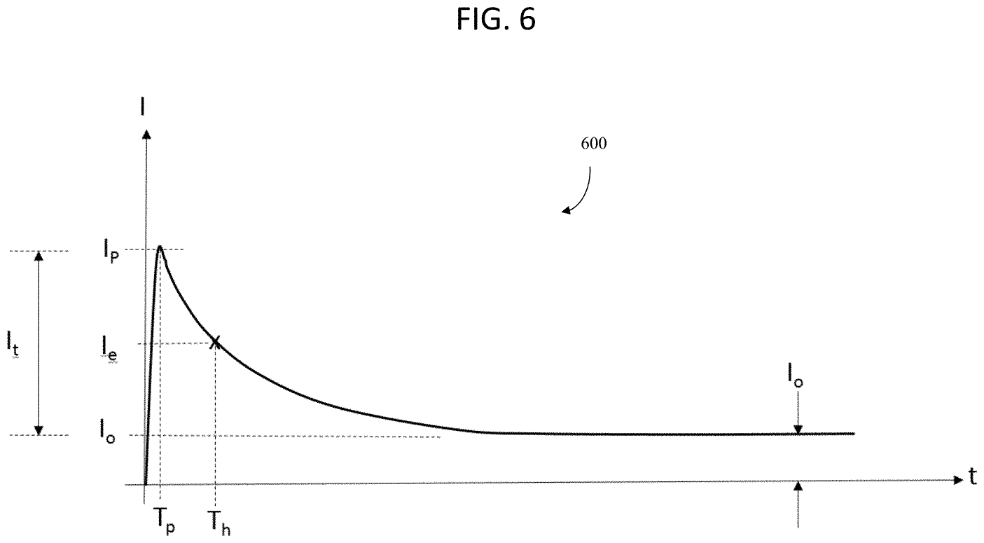

Referring to FIG. 5, the reading of blood from the strip 10 (e.g., between two electrodes of the TIA 304 of FIG. 3) can be modelled as an equivalent circuit 500 depicted in FIG. 5. In the equivalent circuit 500, V is a step response excitation, the blood HCT simplified model consists of a resistor (Ro) which produces a steady state current in time in parallel with the resistance (Rt) and capacitance (Ct) provided in series, which produce a transient current. The step response shown as part of the waveform 600 in FIG. 6 can be generated by the equivalent circuit 500.

Referring to FIG. 6, an example step response waveform 600 that can be generated by the equivalent circuit 500 is depicted. In particular, FIG. 6 depicts a waveform 600 represented on a graph with the y-axis representing current (I) over time (Iₜ) and with the x-axis representing time (t) peak current and the time of the peak (e.g., Iₚ and Tₚ). Additionally, Iₑ is the current at the point that where its value is 1/e (˜36%) of drop from peak. This the time constant for this point. They represent a characteristic of the blood that can be used to determine HCT. The waveform 600 in FIG. 6 represents the current flow through the HCT anode into the HCT cathode to ground and the current through the blood sample that is matched to the equitant circuit. The initial waveform response is dominated by Rt and Ct, yielding a transient spike on the rising edge of the step response and this transient exponentially decays down to the steady state current dominated by Ro, of circuit 500.

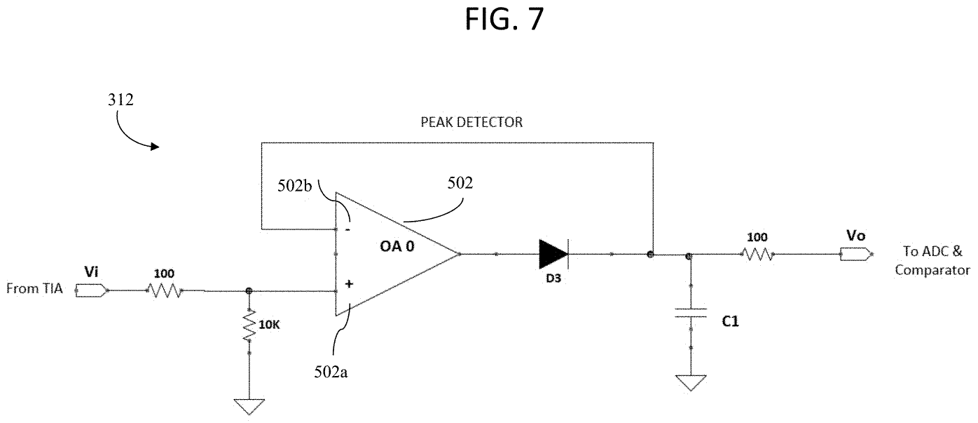

Referring to FIGS. 3 and 7, in some embodiments, the microcontroller 302 include or otherwise be communicatively attached to the test strip 10, the RFB 310 circuit, and a peak detector circuit 312. As would be appreciated by one skilled in the art, although the peak detector circuit 312 and RFB 310 are shown external to the microcontroller 302 in FIG. 3, however in some embodiments, the peak detector circuit 312 and RFB 310 can be integrated on the microcontroller 302 itself. The peak detector circuit 312 can be used to hold the peak value for peak magnitude and as input to peak time comparator 314. For example, the peak detector circuit 312 can be an analog circuit configured to capture the peak current and the time of the peak (e.g., Iₚ and Tₚ in FIG. 6).

In some embodiments, as depicted in FIG. 7, the peak detector 310 can include an operational amplifier 502 configured to receive the voltage (Vi) from the TIA 301 output at the non-inverting input terminal 502a of the operational amplifier 502. The peak detector 310 can also include a Schottky diode (D3) that rectifies the operational amplifier 502 output and which can be fed back into the inverting input terminal 502b operational amplifier 502 as feedback control to form a precision rectifier. The output peak value held steady of the capacitor C1 so it can be read by a slow ADC and the peak detector output value can be cleared or reset by switching the ADC input to a GPIO output and shorting the 100 ohm resistor to ground. At this point in time, the peak detector 312 is ready to detect another peak. In some embodiments, the peak detector 312 can include a combination of resistors and capacitors to control the voltage throughout the circuit. As would be appreciated by one skilled in the art, although FIG. 7 depicts a peak detector 312 with certain resistance values, alternate combinations of resistances can be utilized without departing from the scope of the present disclosure such that the exemplary values in FIG. 7 are provided for exemplary purposes and are not intended to be limiting.

In operation, in some embodiments, the peak detector 312 can be configured to generate a falling edge interrupt at the peak time. In one example, by comparing the 99% of the peak current level to 100% of the transient waveform, there will be a short period of time at the peak when the transient waveform voltage is greater than the peak detected, this time generates the pulse output at the peak. The falling edge interrupt can then be output to an interrupt request input (IRQ) (as depicted in FIG. 3), which will vector an interrupt service routine, that when activated on the peak time, will capture the time to be used to provide the peak time from the excitation rising edge input to the peak current. FIG. 7 illustrates an exemplary example of the peak detector 312.

In operation, in some embodiments, the peak detector 312 can be configured to capture the peak current magnitude of a generated waveform (e.g., waveform 600 depicted in in FIG. 6) and hold the peak value for peak magnitude. This functionality allows ample time for a slow sample rate ADC (e.g., ADC 308) to accurately measure the peak value. The output voltage (Vo) of the peak detector 312 can output to the ADC 308 and a peak time comparator 314. Additionally, the peak detector 312 can provide the peak value for peak magnitude and as input to the peak time comparator 314. By reducing the peak detection by a small amount (e.g., 99%) using the 100/10 k resistors, the output of the peak voltage is guaranteed to be less than the actual transient signal at the very peak, causing the comparator 802 to pulse low at the peak until the transient signal drops below the peak. Capacitor C1 can be provided with a capacitance to hold the peak voltage until it is cleared by the microcontroller 302. For example, a capacitance value of 0.01-1 uF is sufficient. As would be appreciated by one skilled in the art, the values of the resistors in FIG. 7 are for exemplary purposes only and can vary based on the specific implementation without departing from the scope of the present disclosure.

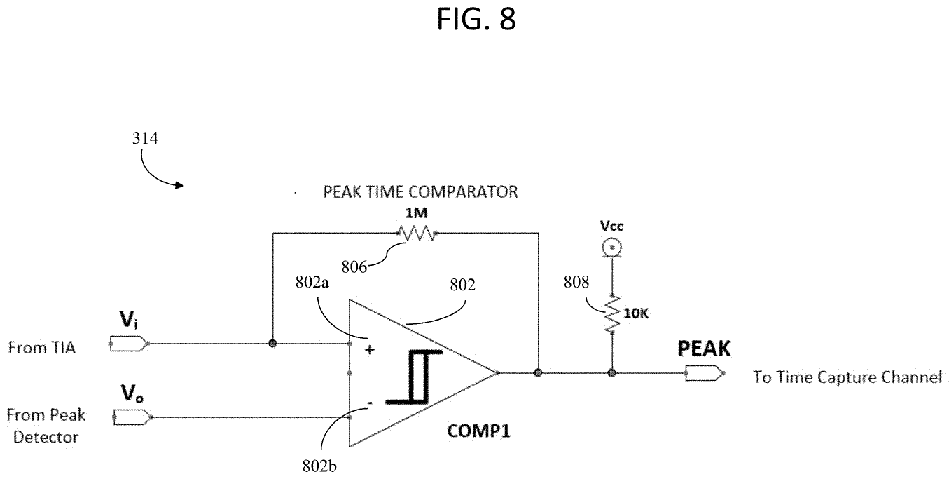

Referring to FIG. 8, an exemplary embodiment of a peak time comparator 314 is depicted. In particular, FIG. 8 depicts an example of a peak time comparator 314 configured to operate within the system 100. In some embodiments, as depicted in FIG. 8, the peak time comparator 314 can include a comparator (COMP1) 802 configured to compare the inputs of Vi from the TIA 304 and Vo from the peak detector 312. The Vi from the TIA 304 can be input into the non-inverting input terminal 802a of the comparator 802 and the Vo from the peak detector 312 can be input into the inverting input terminal 802b of the comparator 802. Additionally, the output of the comparator 802 can be passed through a feedback resistor 806 into the non-inverting input terminal 802a to provide hysteresis so that the output is clean without gitter near the trip point. A value for the feedback resistor 806 can be 10 to 500 times the pullup resistor value to provide a sufficient hysteresis range, but not more than 10 MOhms. As would be appreciated by one skilled in the art, although FIG. 8 depicts a peak time comparator 314 with certain resistance values, alternate combinations of resistances can be utilized without departing from the scope of the present disclosure such that the exemplary values in FIG. 7 are not intended to be limiting. In some embodiments, a high value on the feedback resistor 804 provides a small amount of hysteresis. In some embodiments, the peak time comparator 314 can include a pullup resistor 808 configured to generate a voltage on the output of the comparator 802 because comparators have an open collector output and need a pullup resistor. The value of the pullup resistor 808 can vary, for example, in a value range of 1-10 kOhms. A low value on the output pullup resistor 808 allows for fast edge transitions. As would be appreciated by one skilled in the art, the values of the resistors in FIG. 8 are for exemplary purposes only and can vary based on the specific implementation without departing from the scope of the present disclosure.

In operation, the peak time comparator 314 can be configured to capture the time of the peak current relative to the step input rising edge. The inputs to the peak time comparator 314 are the peak magnitude output from the peak detector (Vo) and the current waveform from a blood sample, such as for example, the real time current waveform 600 response from FIG. 6 (Vi). In some embodiments, the peak comparator 314 can be configured to provide a CLEAR signal to reset the stored peak value for a new reading. Both the peak current detector 312 and peak time comparator 314 are represented in the same circuit, such as for example, within the box labelled “PEAK DET” in FIG. 3.

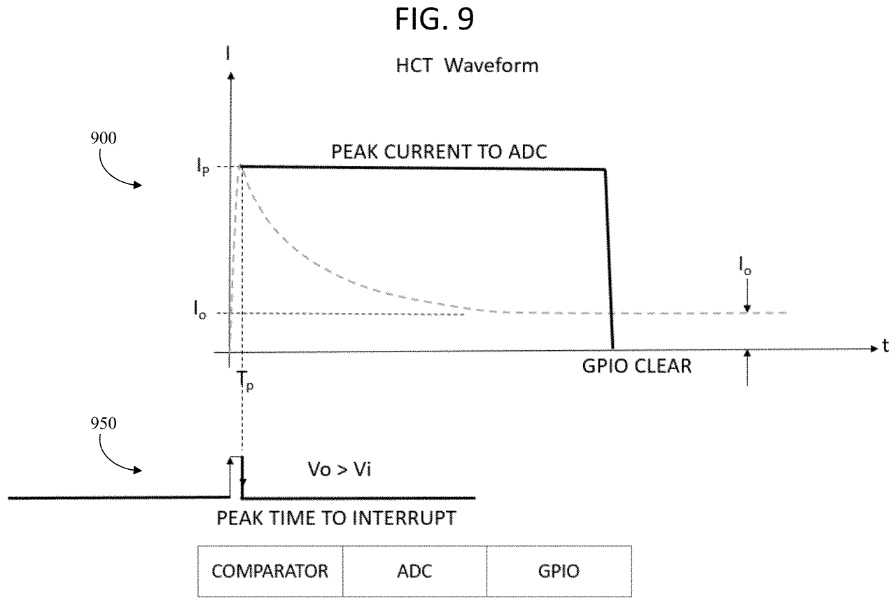

FIG. 9 shows an example HCT current waveform 900 and a peak magnitude current (Iₚ) captured by the peak detector circuit 312 of FIG. 7 along with a peak time falling edge digital signal at (Tₚ) captured by the peak comparator circuit 314 of FIG. 8.

As reflected by the HCT waveform 900 and the DC peak signal 950 depicted in FIG. 9. There are three phases of the measurements provided by the system 100 and reflected in FIG. 9. The phases of measurement can be defined by a “COMPARATOR” phase, an “ADC” phase, and a “General-purpose input/output (GPIO)” phase. In the COMPARATOR phase, the falling edge input triggers a capture/compare interrupt on the microcontroller 302 which captures the interrupt (peak) time. In the ADC phase, the magnitude peak current is measured by an ADC. Finally, in the GPIO phase, after the decay rate has also been captured by the ADC on a separate ADC input, the peak magnitude is cleared by switching the ADC input of the peak detector to a GPIO output LO to clear the peak detector 310 for another cycle. The process may occur multiple times for a single strip 100, in part to stabilize the waveform response.

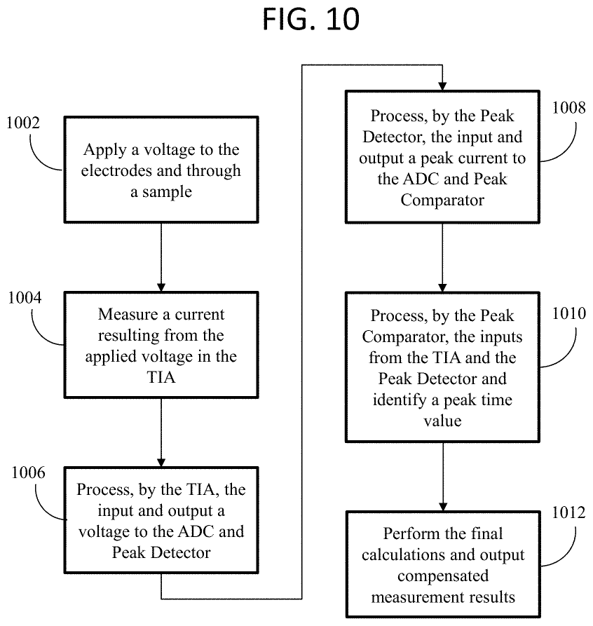

In operation, when a strip 10 with a sample is inserted into a BGM device with the system 300 of the present disclosure, both the peak current and peak time are captured while limiting cost and power to preserve battery life. FIG. 10 depicts an exemplary process 1000 or method for which the system 300 provides the low cost and low power hematocrit measurements for a handheld BGM device. The system 300 can be initialized when a sample (e.g., drop of blood on a test strip) is detected or through another initiation process (e.g., a button press). At step 1002 a voltage step is applied between the electrodes (e.g., HCT electrodes) to generate a current through the sample. At step 1004 the current resulting from the applied voltage is measured by the TIA 304 as a current is applied over the RFB 310. At step 1006 the TIA 304 processes the current inputs and a voltage (Vo) is output by the TIA 304 to the ADC 308 and the peak detector circuit 312.

At step 1008 the peak detector 312 processes the input voltage Vi, and optionally a feedback resistor 310, to identify a peak current and outputs the peak value as a DC voltage output Vo to the ADC 308 and the peak comparator 314. The ADC 308 can measure the DC input of the peak detector 312 output and the output of the ADC 308 can be an internal count value representing the peak current. At step 1010 the peak comparator 314 processes inputs from the TIA 304 and the peak detector 312 and identifies a peak time value for the peak current identified by the peak detector 312. The peak time value is output to the time capture channel for the BGM. Both the peak current and peak time are the key parameters used to map an HCT value for the blood.

At step 1012 the BMG can use the peak current and peak time to calculate an HCT value which is used to compensate for the glucose measurement. In some embodiments, by tabulating values of peak current and peak time over many HCT test samples, these parameters can be used to estimate HCT, for example, either in tabular form or an algorithm based off this data. Once the glucose measurement is complete, the measured HCT value can be used to adjust the glucose measurement. For example, a high HCT value in the blood will appear to show less blood glucose, which must be accounted for and compensated for in the displayed as an HCT compensated glucose results. The HCT compensated glucose results are based on the values received from the TIA 304, the peak detector 312, and the peak comparator 314. The microcontroller 302 will automatically adjust the glucose estimate based on the glucose curve, and the peak HCT and peak HCT time, for example, using a lookup table or an algorithm.

The advantage of the systems and methods of the present disclosure is that it accurately measures the peak current and peak time with a low cost and low power microcontroller (e.g., microcontroller 302) which does not require a very high sampling rate. This technique allows for accurate reconstruction of the HCT parameters at ADC sampling rates much less than the Nyquist rate for the fastest frequency components of the response. This in turn allows for an accurate calculation of the decay rate which is proportional to two of the three equivalent circuit parameters Ct and Rt. The steady state current is easy to measure and yields the final equivalent circuit parameters Ro.

All patents, patent applications, and published references cited herein are hereby incorporated by reference in their entirety. It should be emphasized that the above-described embodiments of the present disclosure are merely possible examples of implementations, merely set forth for a clear understanding of the principles of the disclosure. Many variations and modifications may be made to the above-described embodiment(s) without departing substantially from the spirit and principles of the disclosure. It can be appreciated that several of the above-disclosed and other features and functions, or alternatives thereof, may be desirably combined into many other different systems or applications. All such modifications and variations are intended to be included herein within the scope of this disclosure.

What is claimed is:

1. A system for diagnostic testing comprising:
a test strip; and
an electronic meter for performing a diagnostic test on a sample applied to the test strip inserted therein, the electronic meter comprising:
a housing having a test port for receiving the test strip;
trans-impedance amplifiers (TIAs) configured to read a current over the test strip to generate a response waveform;
an analog-to-digital (ADC) converter to convert the response waveform into a digital value;
a peak detector circuit configured to capture peak current magnitude of the response waveform; and
a peak detector circuit configured to capture a peak current time.

2. The system of claim 1, wherein the reading the current over the test strip includes measuring a response current to an excitation voltage.

3. The system of claim 1, wherein the TIAs generate the response waveform from the current.

4. The system of claim 3, wherein the peak detector circuit captures the peak current time based on the captured peak current magnitude from the peak detector circuit and the response waveform from the TIAs.

5. The system of claim 3, wherein an excitation signal is applied to the blood sample such that the response to the excitation signal is analyzed to determine a glucose concentration in the blood sample.

6. The system of claim 1, wherein the TIAs convert current to a differential voltage waveform for analog-to-digital conversion by the ADC.

7. The system of claim 9, wherein a microprocessor is further programmed to detect when the sample is applied to the test strip.

8. The system of claim 12, wherein a microprocessor is further programmed to display a glucose concentration.

9. A diagnostic testing device comprising:
a housing having a test port for receiving a test strip;
trans-impedance amplifiers (TIAs) configured to read a current over the test strip to generate a response waveform;
a peak detector circuit configured to capture peak current magnitude of the response waveform;
a peak detector circuit configured to capture a peak current time; and
an analog-to-digital (ADC) converter to convert the response waveform and the peak current magnitude into a digital value.

10. The device of claim 9, wherein the reading the current over the test strip includes measuring a response current to an excitation voltage.

11. The device of claim 9, wherein the TIAs generate the response waveform from the current.

12. The device of claim 11, wherein the peak detector circuit captures the peak current time based on the captured peak current magnitude from the peak detector circuit and the response waveform from the TIAs.

13. The device of claim 11, wherein an excitation signal is applied to the blood sample such that the response to the excitation signal is analyzed to determine a glucose concentration in the blood sample.

14. The device of claim 13, wherein the TIAs convert current to a differential voltage waveform for analog-to-digital conversion by the ADC.

15. The device of claim 13, wherein a microprocessor is further programmed to detect when the sample is applied to the test strip.

16. The device of claim 15, wherein a microprocessor is further programmed to display a glucose concentration.

17. A method for a low cost and low power hematocrit testing, the method comprising:
applying a voltage to a sample on a test strip;
measuring, by trans-impedance amplifiers (TIAs), a current through the sample;
processing, by the TIAs, the current and outputting a voltage response waveform to an analog digital converter (ADC);
processing, by the peak detector, the voltage response waveform and outputting a peak current to the ADC; and
calculating and displaying hematocrit compensated glucose results based on the values received by the ADC.

18. The method of claim 17, further comprises:
outputting, by the TIAs, the voltage response waveform to a peak comparator; and
outputting, by the peak detector, the peak current to the peak comparator.

19. The method of claim 18, further comprises processing, by the peak comparator, the voltage response waveform and the peak current and outputting a peak time value.

20. The method of claim 19, calculating the compensated glucose results comprises:
calculating an HTC value based on the peak current and peak time value; and
adjusting a glucose measurement to the hematocrit compensated glucose results.
